(19) United States
(12) Patent Application Publication
(10) Pub. No.: US 2007/0190157 A1
(43) Pub. Date: Aug. 16, 2007

(54) Film lined packaging and method of making same

(75) Inventors: Pradeep Sanghvi, Schererville, IN (US); Garry L. Myers, Kingsport, TN (US); Joseph M. Fuisz, Washington, DC (US); Richard C. Fuisz, McLean, VA (US)

Correspondence Address: HOFFMANN & BARON, LLP, 6900 JERICHO TURNPIKE, SYOSSET, NY, 11791, US

(73) Assignee: MonoSoIRx, LLC.

(21) Appl. No.: 11/655,376

(22) Filed: Jan. 19, 2007

Related U.S. Application Data
(60) Provisional application No. 60/760,438, filed on Jan. 20, 2006.

(51) Int. Cl. A61K 9/14

(52) U.S. Cl. 424/489

(57) ABSTRACT

The present invention relates to packaging in the form of a pouch, which may contain active substances, such as food products, pharmaceutical agents, nutraceuticals and cosmetic agents, or the like. More specifically, in some embodiments, the present invention provides a pouch, which includes at least one porous substrate encompassing a closed volume and at least one water-soluble film at least partially covering the at least one porous substrate. The pouch may contain an active substance within the closed volume, as well as an active substance in the water-soluble film. The present invention also relates to methods of making and using the pouches.

### CROSS-REFERENCE TO RELATED APPLICATIONS

The present application claims the benefit of U.S. Provisional Application No. 60/760,438, filed Jan. 20, 2006, the contents of which are incorporated herein by reference.

### FIELD OF THE INVENTION

The present invention relates to packaging in the form of a pouch, which may contain active substances, such as food products, pharmaceutical agents, nutraceuticals and cosmetic agents or the like. The pouch material may include a water-soluble film covering, which dissolves when the pouch is placed at a selected body site. The present invention also relates to methods of making such pouches, as well as methods of using same.

### BACKGROUND OF THE RELATED TECHNOLOGY

It often is desirable to package drugs, food products and related consumable items into pre-determined amounts. For instance, smokeless tobacco products are conventionally packaged into individual pouches for oral use. Such packaging typically is made from a porous material that is flavorless and insoluble in water. Therefore, the material does not typically dissolve in the mouth during use. The product contained within the pouch, however, flows out through the porous material into the oral cavity during use.

It also is desirable to provide flavors that may be consumed during use of such packaged products. For example, consumers sometimes enjoy experiencing a mint flavor during use of a smokeless tobacco product. Flavorless porous materials, however, have typically been used to form such packages.

Further, undesirable interactions between the packaged product and the porous packaging material sometimes occur in such products. Prior known packaging systems have failed to address this problem.

The present invention, therefore, provides water-soluble film linings, or covers, for porous substrates used in making packaged products, such as pouches. The water-soluble film may contain a flavor that can be experienced along with the edible material housed inside the packaging. Alternatively, the water-soluble film may contain a variety of other active substances for use in combination with an active material housed inside the pouch. The pouches of the present invention thereby overcome the shortcomings of the prior art.

### SUMMARY OF THE INVENTION

In accordance with some embodiments of the present invention, there is provided a pouch for administering an active component, which includes: at least one porous substrate encompassing a closed volume; and at least one water-soluble film at least partially covering the at least one porous substrate.

Some embodiments of the present invention provide a method of making a pouch for administering an active component, which includes the steps of: (a) providing a water-insoluble porous substrate; (b) at least partially covering the porous substrate with a water-soluble film; and (c) folding the at least partially covered porous substrate to define a closed volume.

In some embodiments of the present invention, there is provided a method of delivering multiple active components into the oral cavity of an individual, which includes the steps of:
(a) providing a pouch including:
   (i) at least one porous substrate encompassing a closed volume;
   (ii) at least one water-soluble film at least partially covering the at least one porous substrate, the water-soluble film containing a first active component; and
   (iii) a second active component contained in the closed volume;
(b) applying the pouch into the oral cavity of the individual; and
(c) allowing the at least one water-soluble film to dissolve and release the first active component into the oral cavity of the individual in combination with the second active component.

Some embodiments of the present invention provide a method of delivering an active component in combination with a tobacco product into the oral cavity of an individual, which includes the steps of:
(a) providing a pouch including:
   (i) at least one porous substrate encompassing a closed volume;
   (ii) at least one water-soluble film at least partially covering the at least one porous substrate, the water-soluble film containing an active component; and
   (iii) a tobacco product contained in the closed volume;
(b) applying the pouch into the oral cavity of the individual; and
(c) allowing the at least one water-soluble film to dissolve and release the active component into the oral cavity of the individual in combination with the tobacco product.

### BRIEF DESCRIPTION OF THE DRAWINGS

FIG. 1 is a side elevational view of a pouch in accordance with an embodiment of the present invention;

FIG. 2 is a cross-sectional view taken along line 2-2 of FIG. 1;

FIG. 2a is an alternative cross-sectional view taken along line 2 -2 of FIG. 1;

FIG. 3 is a cross-sectional view of a pouch in accordance with an embodiment of the present invention; and

FIG. 4 is a cross-sectional view taken along line 4-4 of FIG. 3.

### DETAILED DESCRIPTION OF THE INVENTION

The present invention relates to packaging in the form of a pouch, which may be administered at a selected body site, such as within the oral cavity. The pouch may include a porous substrate material, which encompasses a closed volume, and a water-soluble film covering the porous substrate. In some embodiments, the film may be used to line the pouch, whereas in other embodiments, the film may cover the exterior surface of the pouch. Alternatively, the film may cover both the interior and exterior surfaces of the pouch.

A material, such as an edible product, may be contained inside the pouch. Exemplary materials for inclusion inside the pouch include active components, such as food products, pharmaceutical agents, nutraceuticals and cosmetic agents, including flavors, breath fresheners, or the like, but not including tobacco products. Active components also may be incorporated into the water-soluble film used to cover the pouch, such as, for example, flavors or drugs. Upon administration, such as within the oral cavity, the water-soluble film dissolves and releases the active contained therein. The active from the film may commingle with the active contained in the pouch as both active components are released into the oral cavity.

Alternatively, in some embodiments, the material contained inside the pouch and/or incorporated into the water-soluble film may include tobacco products, such as tobacco, tobacco extracts, synthetic compounds of tobacco, tobacco flavors, or the like. Tobacco products may be used instead of active components in any of the embodiments described herein.

In some embodiments, the active housed within the pouch may undesirably interact with the porous substrate. For example, the active may stain or discolor the substrate material. The film-lining, therefore, may provide a barrier for the porous substrate that protects the substrate from the active substance housed within the pouch.

Besides oral administration, a variety of other administration routes are contemplated for the pouches described herein, including but not limited to, buccal, sublingual, transmucosal, periodontal, gingival, nasal, ocular, otic, vaginal, rectal or topical.

As mentioned above, in some embodiments, the pouch may include at least one porous substrate encompassing a closed volume. The pouch also may include at least one water-soluble film, which at least partially covers the porous substrate.

The porous substrate may permit moisture, such as saliva, to flow through the pouch, as well as allowing the enclosed active component or a dissolvable extract thereof to flow out of the pouch into the oral cavity. The porous substrate may include a water-insoluble material, such as those materials conventionally used in smokeless tobacco products, tea bags, or the like. Suitable materials include, but are not limited to, fiber, paper, water-insoluble polymers, cloth and fabric. Water-insoluble polymers such as cellulosic polymers may be used. Specific examples of useful water insoluble polymers include, but are not limited to, ethyl cellulose, hydroxypropyl ethyl cellulose, cellulose acetate phthalate, hydroxypropyl methyl cellulose phthalate and combinations thereof. Composite substrates of various materials, such as those mentioned above, also may be used to form the porous substrate.

In some embodiments, the porous substrate may be at least partially covered by the water-soluble film. The water-soluble film may have a thickness of about 20 micron to about 100 micron. The water-soluble film may dissolve when contacted with moisture at the administration site within the body, such as in the oral cavity. The dissolution rate of the water-soluble film may be adjusted for different embodiments to provide different release rates of the active component contained therein. For example, in some embodiments, the water-soluble film may have a rapid dissolution rate, such as about 1-2 minutes, which provides a rapid release of the active. In other embodiments, the water-soluble film may be adapted to have a slower dissolution rate, such as 30-60 minutes or even up to about 24 hours, which sustains the release of the active component contained in the film. A variety of different factors may affect the dissolution rate of the film, including the film-forming polymers selected and film thickness, among others.

The water-soluble film may include at least one water-soluble polymer. As used herein the phrase “water soluble polymer” and variants thereof refer to a polymer that is at least partially soluble in water, and desirably fully or predominantly soluble in water, or absorbs water.

In some embodiments, the water-soluble polymer may be capable of heat-sealing along with the porous substrate to form a sealed pouch. In addition, different water-soluble polymers or combinations of polymers may be used to adjust the dissolution rate of the film. The dissolution rate also may be adjusted by combining water-soluble polymers having different viscosities or molecular weights.

For instance, in some embodiments, the water-soluble polymer may include polyethylene oxide, alone or in combination with other water-soluble polymers. Water-soluble cellulosic polymers, such as hydroxypropyl cellulose and hydroxypropyl methylcellulose may be employed. Hydroxypropyl methylcellulose, in particular, is capable of heat sealing with the porous substrate material without melting to an undesirable degree.

The molecular weight of polyethylene oxide used in the films may range from about 100,000 to about 8 million. Desirably, the molecular weight of polyethylene oxide ranges from about 100,000 to about 900,000. In addition, blends of different molecular weight polyethylene oxides may be employed, as described in Assignee's co-pending U.S. application Ser. No. 10/856,176 (U.S. Patent Publication No. 2005/0037055 A1), filed on May 28, 2004, the contents of which are incorporated herein by reference in their entirety.

In some embodiments, water-soluble polymers, such as cellulosic polymers, having different viscosities may be used. For example, the water-soluble polymer may include a combination of hydroxypropyl methylcellulose having a viscosity of about 15 cps with hydroxypropyl methylcellulose having a viscosity of about 50 cps. The addition of the higher viscosity hydroxypropyl methylcellulose may impart a slower dissolution rate to the film, such as about 30-60 minutes, which may be desirable in some embodiments. Additionally, the higher viscosity hydroxypropyl methylcellulose may act to encapsulate the active component contained in the film to some degree. Such encapsulation may extend the release of the active over even longer periods of time.

Commercially available examples of such polymers include METHOCEL E15 (hydroxypropyl methylcellulose having an apparent viscosity of 15 cps) and METHOCEL E50 (hydroxypropyl methylcellulose having an apparent viscosity of 50 cps), both available from the Dow Chemical Company.

Examples of other suitable water-soluble polymers for use in the water-soluble films include, but are not limited to, pullulan, hydroxyethyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. The use of such polymers in film are described in detail in U.S. application Ser. No. 10/856,176, referred to above.

In some embodiments, it also may be desirable to add polydextrose to the water-soluble film. Polydextrose is a water-soluble polymer that serves as a filler and solubility enhancer, i.e., it increases the dissolution time of the film, without compromising the sealing properties of the film. Polydextrose may be present in amounts of about 5% to about 30% by weight of film, more specifically 9% to about 15% by weight.

A variety of optional additives also may be included in the water-soluble film, such as, but not limited to, anti-foaming agents, such as silicone-containing compounds, anti-tacking agents, plasticizers, polyalcohols, surfactants and thermo-setting gels such as pectin, carageenan, and gelatin, among others.

Water-soluble films may be prepared by utilizing a selected casting or deposition method and a controlled drying process. Such processes are described in more detail in commonly assigned U.S. application Ser. No. 10/074,272, filed on Feb. 14, 2002, and published as U.S. Patent Publication No. 2003/0107149 A1, the contents of which are incorporated herein by reference in their entirety. Alternatively, water-soluble films may be extruded as described in commonly assigned U.S. application Ser. No. 10/856,176, filed on May 28, 2004, and published as U.S. Patent Publication No. 2005/0037055 A1, the contents of which are incorporated herein by reference in their entirety.

In some embodiments, the water-soluble film itself also may include at least one active component. At least one active component, such as food products, pharmaceutical agents, nutraceuticals or cosmetic agents, also may be contained in the closed volume of the pouch. The active component contained in the water-soluble film may be the same or different from the active housed in the pouch.

In some embodiments, suitable actives for housing in the pouch and/or for incorporation into the water-soluble film include, but are not limited to: food products; botanicals; herbals; minerals; insects; nutraceuticals; pharmaceutical agents; cosmetic agents; drugs; bioactive active substances; medicaments; antidotes; vaccines; antigens or allergens; mouthwash components; flavors; fragrances; enzymes; preservatives; sweetening agents; colorants; spices; vitamins; polyphenols; phytochemicals; and combinations thereof. Such actives do not include tobacco products.

Examples of botanicals include, without limitation: roots; barks; leaves; stems; flowers; fruits; sunflower seeds; and combinations thereof.

A wide variety of medicaments, bioactive active substances and pharmaceutical agents may be included. Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids such as bromocryptine and nicotine, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of medicating active ingredients include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin, acetaminophen, and combinations thereof that may optionally include caffeine.

Other drugs include anti-diarrheals such as immodium AD, anti-histamines, anti-tussives, decongestants, vitamins, and breath fresheners. Suitable vitamins contemplated for use herein include any conventionally known vitamins, such as, but not limited to, Vitamins A, B, C and E. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Also contemplated for use herein are anxiolytics such as alprazolam (available as Xanax®); anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemizole (available as Hismanal™), nabumetone (available as Relafen®), and Clemastine (available as Tavist®); anti-emetics such as granisetron hydrochloride (available as Kytril®)) and nabilone (available as Cesamet™); bronchodilators such as Bentolin®, albuterol sulfate (available as Proventil®); anti-depressants such as fluoxetine hydrochloride (available as Prozac®), sertraline hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and Ca^{H}-antagonists such as nifedipine (available as Procardia® and Adalat®), and verapamil hydrochloride (available as Calan®.

Erectile dysfunction therapies include, but are not limited to, drugs for facilitating blood flow to the penis, and for effecting autonomic nervous activities, such as increasing parasympathetic (cholinergic) and decreasing sympathetic (adrenersic) activities. Useful non-limiting drugs include sildenafils, such as Viagra®, tadalafils, such as Cialis®, vardenafils, apomorphines, such as Uprima®, yohimbine hydrochlorides such as Aphrodyne®, and alprostadils such as Caverject®.

The popular H₂-antagonists that are contemplated for use herein include, but are not limited to, cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

The pharmaceutically active agents may include allergens or antigens, such as, but not limited to, plant pollens from grasses, trees, or ragweed; animal danders, which are tiny scales shed from the skin and hair of cats and other furred animals; insects, such as house dust mites, bees, and wasps; and drugs, such as penicillin.

An anti-oxidant also may be added to prevent the degradation of an active, especially where the active is photosensitive.

The bioactive active substances employed herein may include beneficial bacteria. More specifically, certain bacteria normally exist on the surface of the tongue and in the back of the throat. Such bacteria assist in the digestion of food by breaking down proteins found in the food. It may be desirable, therefore, to incorporate these bacteria into some embodiments of the present invention.

It also may be desirable to include actives for treating breath malodor and related oral care conditions, such as actives which are effective in suppressing microorganisms. Because breath malodor can be caused by the presence of anaerobic bacteria in the oral cavity, which generate volatile sulfur compounds, components that suppress such microorganisms may be desirable. Examples of such components include antimicrobials such as triclosan, chlorine dioxide, chlorates, and chlorites, among others. The use of chlorites, particularly sodium chlorite, in oral care compositions such as mouthrinses and toothpastes is taught in U.S. Pat. Nos. 6,251,372, 6,132,702, 6,077,502, and U.S. Publication No. 2003/0129144, all of which are incorporated herein by reference. Such components are incorporated in amounts effective to treat malodor and related oral conditions.

Cosmetic active agents may include breath freshening compounds like menthol, other flavors or fragrances, especially those used for oral hygiene, as well as actives used in dental and oral cleansing such as quaternary ammonium bases. The effect of flavors may be enhanced using flavor enhancers like tartaric acid, citric acid, vanillin, or the like.

Examples of polyphenols include, without limitation, flavonoids, such as catechins, epicatechins, procyandins and anthocyanins, among others.

Examples of phytochemicals include, without limitation, allyl sulfides, indoles, glucosinolates, sulfaforaphane, isothiocyanates, thiocyanates, thiols, lycopene, carotenoids, phthalides, polyacetylenes, silymarin, monoterpenes, ellagic acid, phenols, flavonoids, phytic acid, saponins, gingerols and glycyrrhizin catechins, among others.

Also color additives may be employed. In some embodiments, it may be desirable to add colorants to the water-soluble film to enhance the overall aesthetic appearance of the pouch. For instance, the active component housed within the pouch may cause undesirable staining of the porous substrates forming the pouch. The film may include a colorant or whitening agent that masks such undesirable staining, thereby improving the appearance of the pouch. Such color additives include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

The flavorings may be added to provide a hot or cold flavored drink or soup. These flavorings include, without limitation, tea and soup flavorings such as beef and chicken.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

Flavors may be present in the water-soluble film in amounts of about 5% to about 30% by weight of the film, more specifically about 15% to about 27% by weight of the film.

Alternatively, in some embodiments, the material housed in the pouch and/or incorporated into the water-soluble film may include one or more tobacco products, such as smokeless tobacco, tobacco extracts, synthetic compounds of tobacco, tobacco flavors, snuff, or the like. Tobacco products also may be used in combination with any of the active components described herein. For instance, a tobacco product may be housed in the closed volume of the pouch and an active component, such as a flavor, may be incorporated into the water-soluble film. Additionally, the water-soluble film may be chopped up and admixed with the tobacco product, in addition to or as an alternative to having the pouch lined with the tobacco product.

Some embodiments also may include an emulsification system in the water-soluble film. An emulsification system may be used to alleviate non-uniform patterns created in the film by flavors, particularly in embodiments incorporating high levels of flavor, such as about 25-30% by weight of the film composition, for an intense flavor impact. Non-uniform patterns may create an adverse film appearance, and thus, may be undesirable in some embodiments. The emulsification system may include any of a variety of emulsifiers, such as, for example, propylene glycol alginate, polyoxyethylene sorbitan monooleate (Polysorbate 80) and/or sorbitan monooleate. In some embodiments, the emulsification system may include propylene glycol alginate in amounts of about 0.5% to about 1.5% by weight of the film, polyoxyethylene sorbitan monooleate in amounts of about 0.1% to about 1% by weight of the film and sorbitan monooleate in amounts of about 0.1% to about 1% by weight of the film.

Actives also may include sweetening agents. The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

In general, the active components contained in the water-soluble film may be present in amounts of about 0.001% to about 30% by weight of the film, more specifically about 1% to about 27% by weight of the film.

In some embodiments, the water-soluble film may include an ionic component to impart or maintain a charged environment to the film. In particular, imparting or maintaining an ionic charge on the surface of the film lining or cover may effect the adhesion properties of the film to the mucosal surfaces. Any component that can impart a net (+) or (−) ionic charge may be used. For instance, acids, bases, salts or any polymers that are capable of imparting an ionic charge may be included in the water-soluble film.

Any of the active components described above may be incorporated into the water-soluble film and/or housed in the closed volume of the pouch. In some embodiments, a different active component may be contained in the pouch from the active component incorporated into the water-soluble film. For example, a flavor may be incorporated into the film and a food product contained in the pouch. Alternatively, some embodiments may include the same active component in the water-soluble film and within the pouch. Additionally, multiple active components may be incorporated into the water-soluble film and/or contained in the pouch.

Suitable active components and details of water-soluble film formation are more fully described in commonly assigned U.S. application Ser. Nos. 10/074,272 and 10/856,176, referred to above, as well as commonly assigned U.S. application Ser. No. 10/768,809, filed on Jan. 30, 2004, the contents of which are incorporated herein by reference in their entirety.

As mentioned above, the water-soluble film may at least partially cover the porous substrate. In some embodiments, the water-soluble film may wholly cover the porous substrate. The at least partially film-covered porous substrate may be formed into a pouch in a variety of different manners.

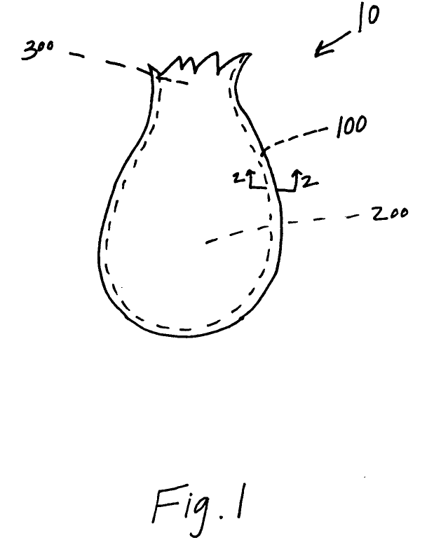

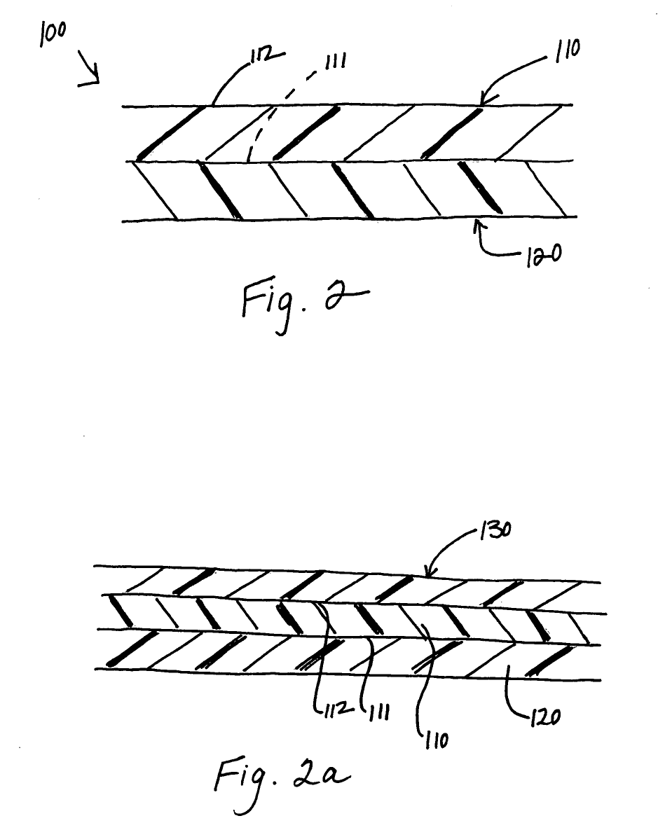

In some embodiments, the porous substrate may be folded such that a closed volume is defined to form a pouch. For example, as shown in FIG. 1, the porous substrate may be folded and gathered into a pouch 10 having pouch wall 100 and enclosing volume 200. The porous substrate may be sealed to itself, such as heat sealed, at the gathering point 300 of the pouch 10. As shown in FIG. 2, taken along the 2-2 axis of FIG. 1, the pouch wall 100 may include a porous substrate 110 having an inner surface 111 and an outer surface 112. The water-soluble film 120 may at least partially cover the inner surface 111 of the substrate 110. Such combination forms a film-lined pouch. Alternatively, the water-soluble film may at least partially cover the outer surface or both the inner and outer surfaces of the porous substrate, as shown in FIG. 2a in which the water-soluble film 130 additionally covers the outer surface 112 of the porous substrate 110.

In some embodiments, the water-soluble film, which at least partially covers the inner and/or outer surfaces of the porous substrate, may be laminated to the surface(s). For example, the water-soluble film may be bonded or adhered to the surface(s).

In an alternative embodiment, two porous substrates may be provided. The two porous substrates may be sheet-like members. As shown in FIG. 3, two porous substrates may be in perimetric face-to-face engagement with one another defining wall 400 and wall 500 of pouch 20 and enclosing volume 600. The porous substrates may be fused to one another at the perimetric face-to-face engagement.

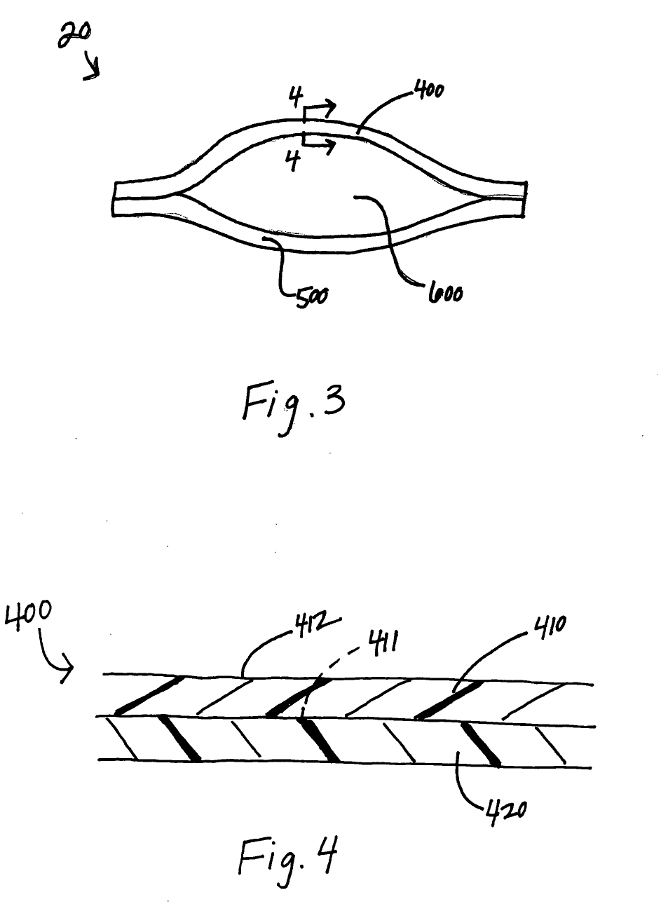

More specifically, as shown in FIG. 4, taken along the 4-4 axis of FIG. 3, wall 400 may include a porous substrate 410 having an inner surface 411 and an outer surface 412. The water-soluble film 420 may at least partially cover the inner surface 411 of the porous substrate 410. Similarly, wall 500 may include a porous substrate having an inner surface and an outer surface and a water-soluble film at least partially covering the inner surface. Such combination forms a film-lined pouch. Alternatively, the water-soluble film may at least partially cover the outer surface or both the inner and outer surfaces of the porous substrates.

A variety of other manners of folding a single porous substrate or multiple porous substrates into a pouch may be employed. For example, a single porous substrate may be folded over itself into a tube-like shape. The tube-like porous substrate may be sealed along its length and at each end to define a closed volume within. The inner and/or outer surfaces of the tube-like porous substrate may be at least partially covered with a water-soluble film. In some embodiments, the water-soluble film may be laminated to the porous substrate. Other manners of folding and sealing the porous substrate(s) are considered well within the scope of the present invention.

The present invention also is directed to methods of making the pouches described above. In accordance therewith, a water-insoluble porous substrate may be provided. The porous substrate may be at least partially covered with a water-soluble film. The water-soluble film may contain any of the various components described above. The porous substrate may have an inner and an outer surface and may be covered with the water-soluble film on either or both surfaces. In some embodiments, the water-soluble film may be laminated to the porous substrate on the inner and/or outer surfaces thereof. For instance, the water-soluble film may be bonded or adhered to the film using, for example, an adhering agent or heat.

Once the porous substrate has been covered with the water-soluble film, it may be folded to define a closed volume, thereby forming a pouch. In some embodiments, the film-covered porous substrate may be gathered or folded over itself and heat-sealed to itself at the points of contact. For example, in some embodiments, a film-covered porous substrate may be folded over itself such that one portion of the substrate is engaged along the perimeter with a second portion of the substrate. The substrate may be heat-sealed at the perimetric points of engagement. In other embodiments, for example, two film-covered porous substrates, which are in perimetric face-to-face engagement, may be fused or heat-sealed to one another along the perimeter. In some embodiments, the water-soluble film may be heat-sealed with the porous substrate.

Prior to heat sealing the pouch, an active component may be positioned within the closed volume defined therein. Any of the active components described above may be housed in the pouch.

In some embodiments, for example, the at least partially film-covered porous substrate may be folded over itself to form a pouch having a closed volume. Two sides of the pouch may be sealed closed, leaving one side of the pouch open. An active component or a tobacco product may be filled into the closed volume via the open side of the pouch. The open side of the pouch then may be sealed closed to form the final product. For instance, the sides of the pouch may be sealed by heat and/or pressure. Alternatively, in some embodiments, a strand of pouches may be formed in which one side of the strand of pouches is open. A portion of an active component or a tobacco product may be filled into each pouch. Subsequently, the open side of the strand of pouches may be sealed closed and individual pouches may be produced by severing them from the strand. This process is described in more detail in U.S. Pat. No. 5,174,088 to Focke et al., which is incorporated herein by reference in its entirety.

The present invention also is directed to methods of delivering multiple active components into the oral cavity of an individual. In accordance with such methods, a pouch may be provided. The pouch may include at least one porous substrate encompassing a closed volume. In addition, at least one water-soluble film may at least partially cover the porous substrate. The water-soluble film may include a first active component. The water-soluble film also may include any of the other components described above. A second active component may be contained in the closed volume of the pouch. The first and second active components may be the same or different. The pouch then may be applied into the oral cavity of an individual. Once applied into the oral cavity, and as saliva begins to mix with the pouch, the water-soluble film may be allowed to dissolve and release the first active component into the oral cavity of the individual. Desirably, the second active component may release from the pouch into the oral cavity as well, in combination with the first active component.

More specifically, in some embodiments, as the first active component releases from the water-soluble film, it may combine with the second active component housed in the pouch. A portion of the first active component may be sorbed by the second active component as it is released from the water-soluble film. The sorbed concentration of the first active component may increase as more film dissolves. Then, as saliva mixes with the pouch and reaches the enclosed second active component, a portion of the first active sorbed in the second active also may mix with the saliva and release from the pouch. Such mechanism may provide an extended release of the first active component into the oral cavity of the individual. For instance, if the first active component is a flavor, this mechanism may provide an extended flavor release throughout the product use. Moreover, the sorption of the first active component may be manipulated by varying the moisture content of the second active component housed in the pouch.

Alternatively, methods are provided for delivering an active component in combination with a tobacco product into the oral cavity of an individual. Similar to above, a pouch may be provided. The pouch may include at least one porous substrate encompassing a closed volume. In addition, at least one water-soluble film may at least partially cover the porous substrate. The water-soluble film may include an active component. The water-soluble film also may include any of the other components described above. A tobacco product may be contained in the closed volume of the pouch. The pouch may be applied into the oral cavity of an individual. Once applied into the oral cavity, and as saliva begins to mix with the pouch, the water-soluble film may be allowed to dissolve and release the active component into the oral cavity of the individual. Desirably, the tobacco product may release from the pouch into the oral cavity as well, in combination with the active component.

### EXAMPLES

### Example 1

Film-lined pouches of the present invention are prepared in accordance with the following. Water-soluble films for use in covering the porous substrates of the pouches are prepared using the amounts described in Table 1.

TABLE 1

| Component | Weight % |
|---|---|
| Hydroxypropyl methylcellulose (15 cps) | 34.69 |
| Hydroxypropyl methylcellulose (50 cps) | 8.00 |
| Polyethylene oxide | 7.15 |
| Polydextrose | 10.14 |
| Propylene glycol alginate¹ | 1.00 |
| Glycerol monooleate² | 1.00 |
| Polysorbate 80³ | 0.30 |
| Sorbitan monooleate⁴ | 0.20 |
| Propylene glycol | 3.00 |
| Glycerin | 3.00 |
| Amorphous precipitated silica⁵ | 1.00 |
| Magnesium stearate | 0.50 |
| Methyl paraben | 0.02 |
| Sucralose | 2.00 |
| Flavor | 27.00 |
| Hydrophilic titanium dioxide | 1.00 |

¹Commercially available as Colloid 602
²Commercially available as ALDO MO
³Commercially available as T SOL P-80
⁴Commercially available as Crill 4 NF
⁵Commercially available as Sipernat from Degussa (or SAPS FK500LS)

Water is added to a beaker with the glycerol monooleate, Polysorbate 80, sorbitan monooleate, propylene glycol and glycerin. The beaker is secured on a hot plate with a clamp. Agitation is initiated with a mixing blade of a mixer apparatus and the propylene glycol alginate, titanium dioxide and methyl paraben are slurried into the batch. Mixing continues for 10 minutes. The batch is heated to 85° C. and then the hydroxypropyl methylcellulose (15 cps) is slurried in, followed by the hydroxypropyl methylcellulose (50 cps). The batch is mixed until dispersed evenly. The polyethylene oxide is slurried into the batch and mixed until dispersed evenly. The polydextrose and sucralose are slurried into the batch and mixed until dispersed evenly. Agitation is ceased and the silica and magnesium stearate are added to the batch. Agitation is initiated again at a low speed (setting 1). Mixing continues for 5 minutes and then the batch is removed from the heat. As the solution begins to gain viscosity (thicken), the agitation speed is slowly lowered to allow the mix to cool quicker. Once the solution reaches room temperature, it is mixed on first gear (setting 3). Mixing is continued until the polymers are hydrated. The solution is removed from the mixer and split into four 200 gram batches.

A different flavor combination is added to each of the four batches. The flavor combination added to the first batch is Dr. Pepper type flavor, cherry flavor and kola flavor. The flavor combination added to the second batch is kola flavor and cherry flavor. The flavor combination added to the third batch is Dr. Pepper type flavor and vanilla flavor. The flavor combination added to the fourth batch is kola flavor, vanilla flavor and brown sugar flavor.

After the individual flavor combinations are added to the four batches, each batch is mixed on high agitation for about 10 minutes. Then each batch is mixed on low agitation (setting 2) for 5 minutes. The mixer is switched to first gear and each batch is mixed on setting 2 until ready to use.

Each batch is cast into film and dried. Subsequently, each film is cut into pieces suitable for use in forming a pouch of the present invention. Porous substrates are provided and the film pieces are positioned such that the films at least partially cover the porous substrate. The film pieces may be laminated to the porous substrate on one or both sides of the substrate. The film may be laminated to the substrate by heat and/or pressure. The substrate then is folded over itself to form a pouch having a closed volume. Two sides of the pouch may be sealed at the points of contact, leaving one side of the pouch open. An active component or a tobacco product then may be filled into the pouch via the open side. The filling portion of the active component or the tobacco product may be metered out by weight. The open side then may be sealed closed, for instance, by application of heat and/or pressure, to form the filled pouch.

Alternatively, a strand of individual pouches may be formed. First, a length of film-covered porous substrate may be folded over itself. A number of individual pockets may be defined therein by forming seams between each pocket along the length of the strand, and leaving the strand open on one side. The seams between the pockets may be formed by application of heat and/or pressure. A portion of an active component or a tobacco product then may be metered into each pocket via the open side. The open side then may be sealed along the entire strand by heat and/or pressure. Individual pouches may be obtained by severing each pocket from the strand.

A number of individual filled pouches may be packaged into a container, e.g., a can, to be sold for consumption.

### Example 2

Film-lined pouches of the present invention are prepared in accordance with the following. Water-soluble films for use in covering the porous substrates of the pouches are prepared using the amounts described in Table 2.

TABLE 2

| Component | Weight % |
|---|---|
| Hydroxypropyl methylcellulose (15 cps) | 32.50 |
| Hydroxypropyl methylcellulose (50 cps) | 8.20 |
| Polyethylene oxide | 7.50 |
| Polydextrose | 9.78 |
| Propylene glycol alginate¹ | 1.00 |
| Glycerol monooleate² | 1.00 |
| Polysorbate 80³ | 0.30 |
| Sorbitan monooleate⁴ | 0.20 |
| Propylene glycol | 5.00 |
| Glycerin | 5.00 |
| Amorphous precipitated silica⁵ | 1.00 |
| Magnesium stearate | 0.50 |
| Methyl paraben | 0.02 |
| Sucralose | 2.00 |
| Flavor | 25.00 |
| Hydrophilic titanium dioxide | 1.00 |

¹Commercially available as Colloid 602
²Commercially available as ALDO MO
³Commercially available as T SOL P-80
⁴Commercially available as Crill 4 NF
⁵Commercially available as Sipernat from Degussa (or SAPS FK500LS)

Water is added to a beaker with the glycerol monooleate and heated to 85-90° C. A blend of the methyl paraben and titanium dioxide is added to the batch and dispersed therein for about 10 minutes. A blend of the hydroxypropyl methylcellulose (15 cps), hydroxypropyl methylcellulose (50 cps), polyethylene oxide, sucralose, silica, magnesium stearate and polydextrose are added to the batch. Then the propylene glycol alginate, propylene glycol, glycerin, sorbitan monooleate and Polysorbate 80 are added to the batch. The heat is removed when the polymers are well dispersed in the batch and more water is added to cool the batch. The solution is split into four 200 gram batches.

When the temperature reaches about 50° C., the flavor is added to each batch and the polymers are allowed to hydrate. A different flavor is added to each of the four batches. Orange is added to the first batch. Mandarin orange is added to the second batch. Cappuccino is added to the third batch. Cinnamon is added to the fourth batch.

After the flavors are added to the four batches, each batch is mixed on high agitation for about 10 minutes. Then each batch is mixed on low agitation (setting 2) for 5 minutes. The mixer is switched to first gear and each batch is mixed on setting 2 until ready to use.

Each batch is cast into film and dried. Subsequently, film-lined pouches are prepared as described in Example 1.

### Example 3

Film-lined pouches of the present invention are prepared in accordance with the following. Water-soluble films for use in covering the porous substrates of the pouches are prepared using the amounts described in Table 3.

TABLE 3

| Component | Weight % |
|---|---|
| Hydroxypropyl methylcellulose (15 cps) | 38.00 |
| Hydroxypropyl methylcellulose (50 cps) | 10.00 |
| Polyethylene oxide | 9.00 |
| Polydextrose | 11.98 |
| Glycerol monooleate¹ | 1.00 |
| Polysorbate 80² | 0.30 |
| Sorbitan monooleate³ | 0.20 |
| Propylene glycol | 5.00 |
| Glycerin | 5.00 |
| Amorphous precipitated silica⁴ | 1.00 |
| Magnesium stearate | 0.50 |
| Methyl paraben | 0.02 |
| Sucralose | 2.00 |
| Flavor | 15.00 |
| Hydrophilic titanium dioxide | 1.00 |

¹Commercially available as ALDO MO
²Commercially available as T SOL P-80
³Commercially available as Crill 4 NF
⁴Commercially available as Sipernat from Degussa (or SAPS FK500LS)

Water is added to a beaker with the glycerol monooleate and heated to 85-90° C. A blend of the methyl paraben and titanium dioxide is added to the batch and dispersed therein for about 10 minutes. A blend of the hydroxypropyl methylcellulose (15 cps), hydroxypropyl methylcellulose (50 cps), polyethylene oxide, sucralose, silica, magnesium stearate and polydextrose are added to the batch. Then the propylene glycol, glycerin, sorbitan monooleate and Polysorbate 80 are added to the batch. The heat is removed when the polymers are well dispersed in the batch and more water is added to cool the batch. The solution is split into four 100 gram batches.

When the temperature reaches about 50° C., the flavor is added to each batch and the polymers are allowed to hydrate. Flavors are added to each of the four batches. Mocha is added to the first batch. Orange cognac is added to the second batch. Wintergreen is added to the third and fourth batches.

After the flavors are added to the four batches, each batch is mixed on high agitation for about 10 minutes. Then each batch is mixed on low agitation (setting 2) for 5 minutes. The mixer is switched to first gear and each batch is mixed on setting 2 until ready to use.

Each batch is cast into film and dried. Subsequently, film-lined pouches are prepared as described in Example 1.

### Example 4

Film-lined pouches of the present invention are prepared in accordance with the following. Water-soluble films for use in covering the porous substrates of the pouches are prepared using the amounts described in Table 4.

TABLE 4

| Component | Weight % |
|---|---|
| Hydroxypropyl methylcellulose (15 cps) | 35.00 |
| Hydroxypropyl methylcellulose (50 cps) | 9.20 |
| Polyethylene oxide | 8.30 |
| Polydextrose | 11.98 |
| Glycerol monooleate¹ | 1.00 |
| Propylene glycol | 5.00 |
| Glycerin | 5.00 |
| Amorphous precipitated silica² | 1.00 |
| Magnesium stearate | 0.50 |
| Methyl paraben | 0.02 |
| Sucralose | 2.00 |
| Flavor | 20.00 |
| Hydrophilic titanium dioxide | 1.00 |

¹Commercially available as ALDO MO
²Commercially available as Sipernat from Degussa (or SAPS FK500LS)

Water is added to a beaker with the glycerol monooleate and heated to 85-90° C. A blend of the methyl paraben, silica, magnesium stearate, sucralose and titanium dioxide is added to the batch and dispersed therein for about 10 minutes. A blend of the hydroxypropyl methylcellulose (15 cps), hydroxypropyl methylcellulose (50 cps), polyethylene oxide and polydextrose is added to the batch. Then the propylene glycol and glycerin are added to the batch. The heat is removed when the polymers are well dispersed in the batch and more water is added to cool the batch. The solution is split into seven 50 gram batches.

When the temperature reaches about 50° C., the flavor is added to each batch and the polymers are allowed to hydrate. Flavors are added to each of the seven batches. Cappuccino is added to the first batch. Mocha is added to the second batch. Mandarin orange is added to the third batch. Orange is added to the fourth batch. Orange cognac is added to the fifth batch. Wintergreen is added to the sixth and seventh batches.

After the flavors are added to the seven batches, each batch is mixed on high agitation for about 10 minutes. Then each batch is mixed on low agitation (setting 2) for 5 minutes. The mixer is switched to first gear and each batch is mixed on setting 2 until ready to use.

Each batch is cast into film and dried. Subsequently, film-lined pouches are prepared as described in Example 1.

### Example 5

Film-lined pouches of the present invention are prepared in accordance with the following. Water-soluble films for use in covering the porous substrates of the pouches are prepared using the amounts described in Table 5.

TABLE 5

| Component | Weight % |
|---|---|
| Hydroxypropyl methylcellulose (15 cps) | 38.00 |
| Hydroxypropyl methylcellulose (50 cps) | 10.00 |
| Polyethylene oxide | 9.00 |
| Polydextrose | 12.98 |
| Propylene glycol | 5.00 |
| Glycerin | 5.00 |
| Amorphous precipitated silica¹ | 1.00 |
| Antifoaming agent | 0.01 |
| Methyl paraben | 0.02 |
| Sucralose | 2.00 |
| Peppermint flavor | 15.99 |
| Hydrophilic titanium dioxide | 1.00 |

¹Commercially available as Sipernat from Degussa (or SAPS FK500LS)

Water is added to a beaker with the antifoaming agent and heated to 85-90° C. A blend of the methyl paraben, silica, sucralose and titanium dioxide is added to the batch and dispersed therein. A blend of the hydroxypropyl methylcellulose (15 cps), hydroxypropyl methylcellulose (50 cps), polyethylene oxide and polydextrose is added to the batch. Then the propylene glycol and glycerin are added to the batch. The heat is removed when the polymers are well dispersed in the batch and more water is added to cool the batch. When the temperature reaches about 50° C., the peppermint flavor is added and the polymers are allowed to hydrate.

The batch is mixed on high agitation for about 10 minutes, then low agitation (setting 2) for 5 minutes. The mixer is switched to first gear and the batch is mixed on setting 2 until ready to use.

The batch is cast into film and dried. Subsequently, film-lined pouches are prepared as described in Example 1.

What is claimed is:

1. A pouch for administering an active component, comprising:
at least one porous substrate encompassing a closed volume; and
at least one water-soluble film at least partially covering said at least one porous substrate.

2. The pouch of claim 1, wherein said at least one porous substrate comprises a water-insoluble material.

3. The pouch of claim 2, wherein said water-insoluble material is selected from the group consisting of: fiber; paper; water-insoluble polymers; cloth; and fabric.

4. The pouch of claim 1, wherein said water-soluble film comprises at least one water-soluble polymer.

5. The pouch of claim 4, wherein said water-soluble polymer is capable of heat-sealing.

6. The pouch of claim 4, wherein said water-soluble polymer is selected from the group consisting of: hydroxypropyl methylcellulose; polyethylene oxide; and combinations thereof.

7. The pouch of claim 4, wherein said water-soluble polymer comprises hydroxypropyl methylcellulose having a viscosity of about 15 cps and hydroxypropyl methylcellulose having a viscosity of about 50 cps.

8. The pouch of claim 6, further comprising polydextrose.

9. The pouch of claim 1, wherein said water-soluble film comprises at least one active component.

10. The pouch of claim 9, wherein said active component is selected from the group consisting of: food products; botanicals; herbals; minerals; insects; nutraceuticals; pharmaceutical agents; cosmetic agents; drugs; medicaments; antidotes; vaccines; antigens or allergens; mouthwash components; flavors; fragrances; enzymes; preservatives; sweetening agents; colorants; spices; vitamins; and combinations thereof.

11. The pouch of claim 10, wherein said colorant comprises a whitening agent.

12. The pouch of claim 1, wherein said water-soluble film has a dissolution rate of about 1 minute to about 2 minutes.

13. The pouch of claim 1, wherein said water-soluble film has a dissolution rate of about 30 minutes to about 60 minutes.

14. The pouch of claim 1, wherein said water-soluble film has a dissolution rate of up to about 24 hours.

15. The pouch of claim 1, further comprising an active component contained in said closed volume.

16. The pouch of claim 15, wherein said active component is selected from the group consisting of: food products; botanicals; herbals; minerals; insects; nutraceuticals; pharmaceutical agents; cosmetic agents; drugs; medicaments; antidotes; vaccines; antigens or allergens; mouthwash components; flavors; fragrances; enzymes; preservatives; sweetening agents; colorants; spices; vitamins; and combinations thereof

17. The pouch of claim 1, further comprising at least one tobacco product contained in said closed volume.

18. The pouch of claim 1, wherein said at least one porous substrate has an inner surface and said water-soluble film is at least partially covering said inner surface.

19. The pouch of claim 18, wherein said water-soluble film is laminated to said inner surface.

20. The pouch of claim 1, wherein said at least one porous substrate has an outer surface and said water-soluble film is at least partially covering said outer surface.

21. The pouch of claim 20, wherein said water-soluble film is laminated to said outer surface.

22. The pouch of claim 1, wherein first and second porous substrates are provided, wherein said first porous substrate comprises a sheet-like member and said second porous substrate comprises a sheet-like member, said first and second porous substrates being in perimetric face-to-face engagement with one another.

23. The pouch of claim 22, wherein said first porous substrate and said second porous substrate are fused at said perimetric face-to-face engagement.

24. The pouch of claim 22, wherein said first porous substrate has a first inner surface and said second porous substrate has a second inner surface, wherein said water-soluble film is at least partially covering said first inner surface and a second water-soluble film is at least partially covering said second inner surface.

25. The pouch of claim 22, wherein said first porous substrate has a first outer surface and said second porous substrate has a second outer surface, wherein said water-soluble film is at least partially covering said first outer surface and a second water-soluble film is at least partially covering said second outer surface.

26. The pouch of claim 1, wherein one substrate is provided, said substrate being folded to define said closed volume.

27. The pouch of claim 1, wherein said water-soluble film has a thickness of about 20 micron to about 1000 micron.

28. The pouch of claim 1, wherein said water-soluble film comprises an anti-foaming agent.

29. The pouch of claim 1, wherein said water-soluble film comprises a flavor present in amounts of about 5% to about 27% by weight of said film.

30. The pouch of claim 29, wherein said water-soluble film further comprises an emulsification system, said emulsification system comprising propylene glycol alginate, polyoxyethylene sorbitan monooleate and sorbitan monooleate.

31. The pouch of claim 1, wherein said water-soluble film is extruded.

32. The pouch of claim 1, wherein said water-soluble film further comprises an ionic component that imparts or maintains a charged environment to the water-soluble film.

33. A method of making a pouch for administering an active component, comprising the steps of:
(a) providing a water-insoluble porous substrate;
(b) at least partially covering the porous substrate with a water-soluble film; and
(c) folding the at least partially covered porous substrate to define a closed volume.

34. The method of claim 33, further comprising the step of heat-sealing the at least partially covered porous substrate to itself.

35. A method of delivering multiple active components into the oral cavity of an individual, comprising the steps of:
(a) providing a pouch comprising:
(i) at least one porous substrate encompassing a closed volume;
(ii) at least one water-soluble film at least partially covering the at least one porous substrate, said water-soluble film comprising a first active component; and
iii) a second active component contained in the closed volume;
(b) applying the pouch into the oral cavity of the individual; and
(c) allowing the at least one water-soluble film to dissolve and release the first active component into the oral cavity of the individual in combination with the second active component.

36. The method of claim 35, wherein said first active component comprises a flavor and said second active component is selected from the group consisting of food products, pharmaceutical agents, nutraceuticals and cosmetic agents.

37. A method of delivering an active component in combination with a tobacco product into the oral cavity of an individual, comprising the steps of:
(a) providing a pouch comprising:
(i) at least one porous substrate encompassing a closed volume;
(ii) at least one water-soluble film at least partially covering the at least one porous substrate, said water-soluble film comprising an active component; and
(iii) a tobacco product contained in the closed volume;
(b) applying the pouch into the oral cavity of the individual; and
(c) allowing the at least one water-soluble film to dissolve and release the active component into the oral cavity of the individual in combination with the tobacco product.
